(19) United States
(12) Patent Application Publication
(10) Pub. No.: US 2025/0384980 A1
(43) Pub. Date: Dec. 18, 2025

(54) AI-Powered Personalized Weight Management Systems and Methods

(71) Applicants: Ashish Atreja, Sacramento, CA (US); Aarit Atreja, Sacramento, CA (US); Anvi Atreja, Sacramento, CA (US)

(72) Inventors: Ashish Atreja, Sacramento, CA (US); Aarit Atreja, Sacramento, CA (US); Anvi Atreja, Sacramento, CA (US)

(21) Appl. No.: 19/241,331

(22) Filed: Jun. 17, 2025

Related U.S. Application Data
(60) Provisional application No. 63/660,923, filed on Jun. 17, 2024.

(51) Int. Cl. G16H 20/00; G16H 10/20; G16H 10/60; G16H 40/67; G16H 50/70; G16H 80/00

(52) U.S. Cl. CPC G16H 20/00; G16H 10/20; G16H 10/60; G16H 40/67; G16H 50/70; G16H 80/00

(57) ABSTRACT

AI-powered personalized weight management systems and methods that identify the most evidence-based treatment options for individuals through an online or automatic/bot screener which matches the individuals to a marketplace of proven solutions. Embodiments use continuous engagement that integrate real-world and clinical trial data from devices, software, and coaching services to continuously refine the best next steps for a patient in their weight, cardiometabolic and lifestyle management journey for aiding in effective and sustainable approaches to lifelong health weight management.

### CROSS-REFERENCES TO RELATED PATENT APPLICATIONS

This application claims benefit under U.S. Provisional Patent Application No. 63/660,923, filed on Jun. 17, 2024, which is hereby specifically incorporated by reference in its entirety into the present disclosure.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### FIELD

The field of the inventive subject matter relates to healthcare technology and, more specifically, to systems and methods for weight management utilizing Artificial Intelligence (AI) algorithms and continuous patient engagement strategies.

### BACKGROUND

Current solutions for weight, cardiometabolic and lifestyle management include dietary programs, exercise regimens, pharmaceuticals, and surgical interventions. Some systems also use technology for tracking physical metrics and providing diet plans. However, these solutions are limited in that they often lack personalized treatment options and suffer from low patient engagement rates. Moreover, the existing solutions are usually isolated and do not provide an integrated approach that adapts over time based on patient data and feedback.

### SUMMARY

The illustrative examples also referred to as embodiments provide for AI-powered personalized weight, cardiometabolic and lifestyle management systems and methods that identify the most evidence-based treatment options for individuals through an online or automatic/bot screener and matches them to a marketplace of proven solutions. Furthermore, embodiments or examples of these systems and methods incorporate novel systems that allow for continuous engagement that integrate data from devices, software, and coaching services to continuously refine the best next steps for a patient in their evidence-based weight, cardiometabolic and lifestyle management journey. These embodiments aid in effective and sustainable approaches to lifelong health weight management.

### DETAILED DESCRIPTION OF THE DRAWINGS

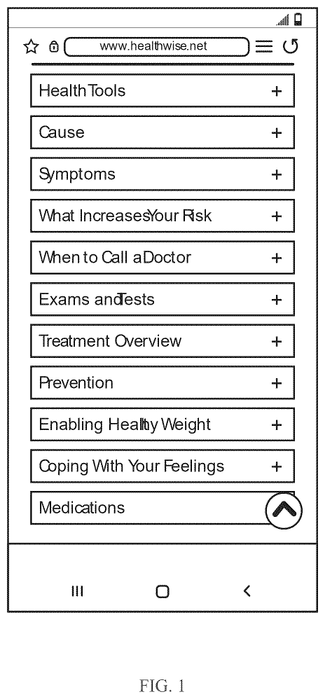

FIG. 1 illustrates an AI-powered BOT using evidence from hundreds of thousands of published literature and guided education and recommendations in accordance with embodiments of the inventive subject matter;

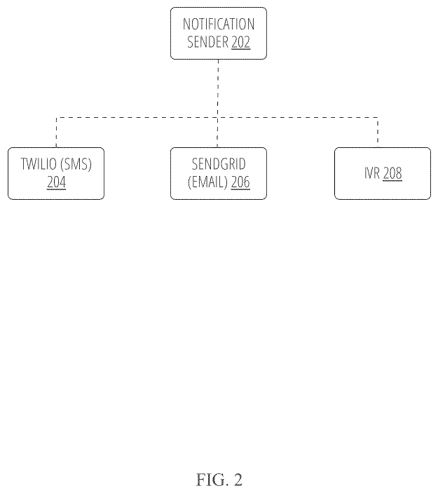

FIG. 2 illustrates an algorithmic workflow for personalized treatment identification and continuous engagement in accordance with embodiments of the inventive subject matter;

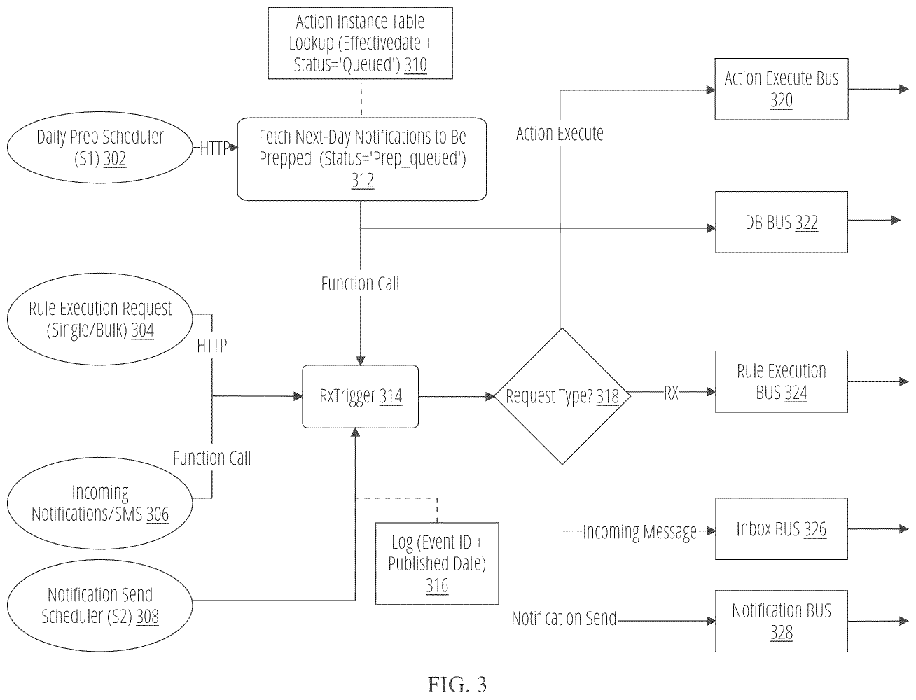

FIG. 3 illustrates the algorithmic workflow for personalized treatment identification and continuous engagement in accordance with embodiments of the inventive subject matter;

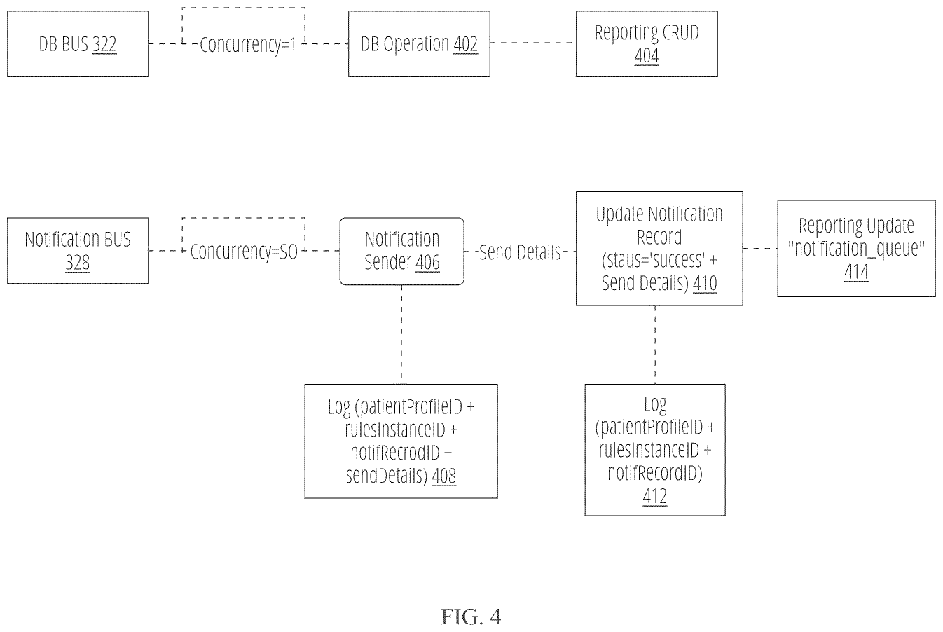

FIG. 4 illustrates the algorithmic workflow for personalized treatment identification and continuous engagement in accordance with embodiments of the inventive subject matter;

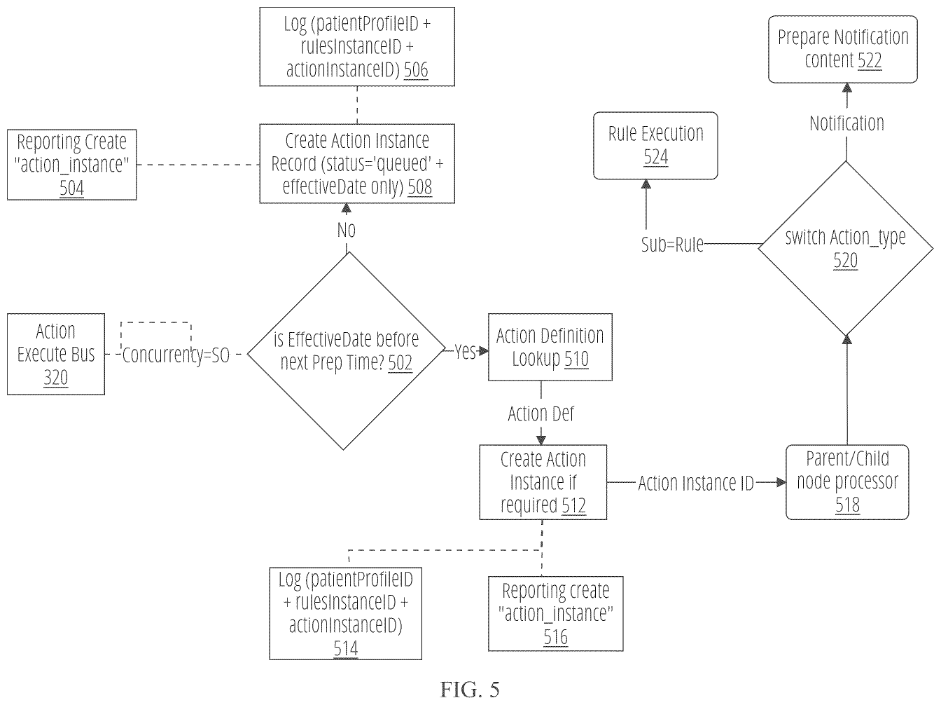

FIG. 5 illustrates the algorithmic workflow for personalized treatment identification and continuous engagement in accordance with embodiments of the inventive subject matter;

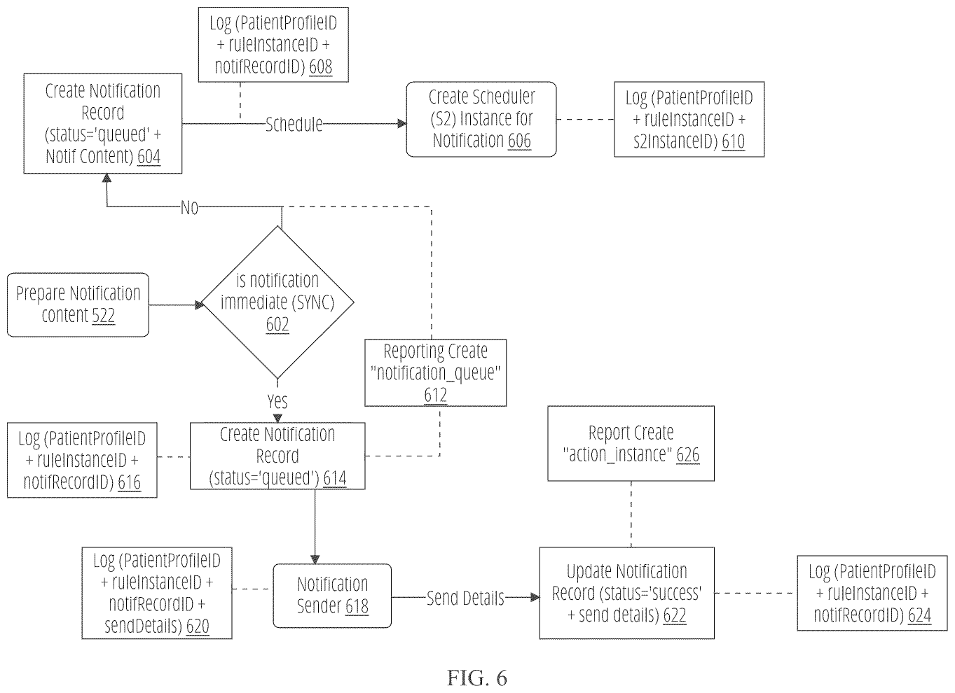

FIG. 6 illustrates the algorithmic workflow for personalized treatment identification and continuous engagement in accordance with embodiments of the inventive subject matter;

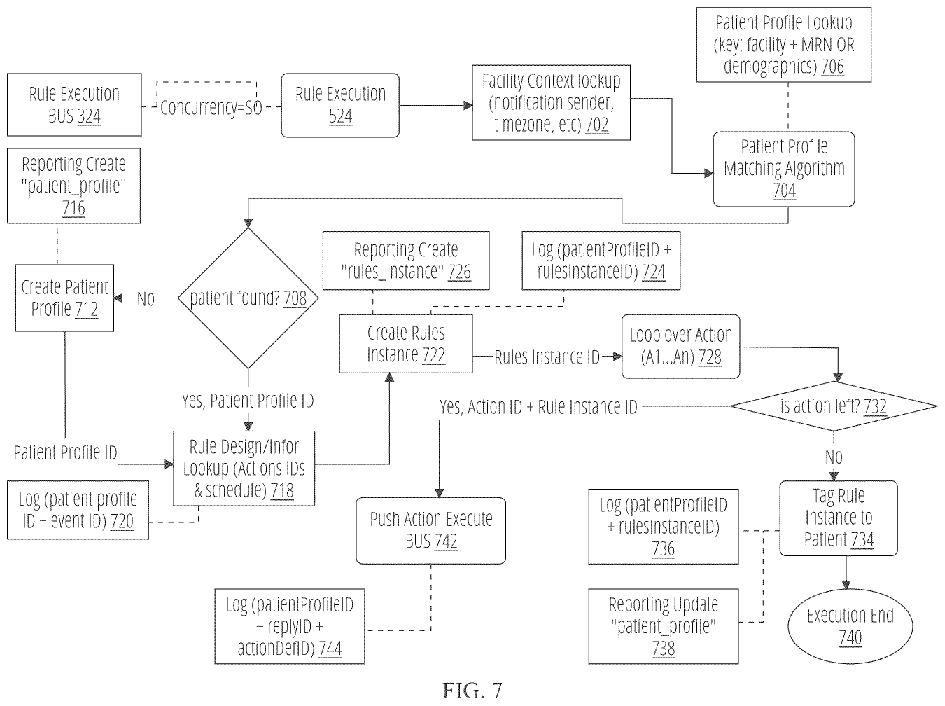

FIG. 7 illustrates the algorithmic workflow for personalized treatment identification and continuous engagement in accordance with embodiments of the inventive subject matter;

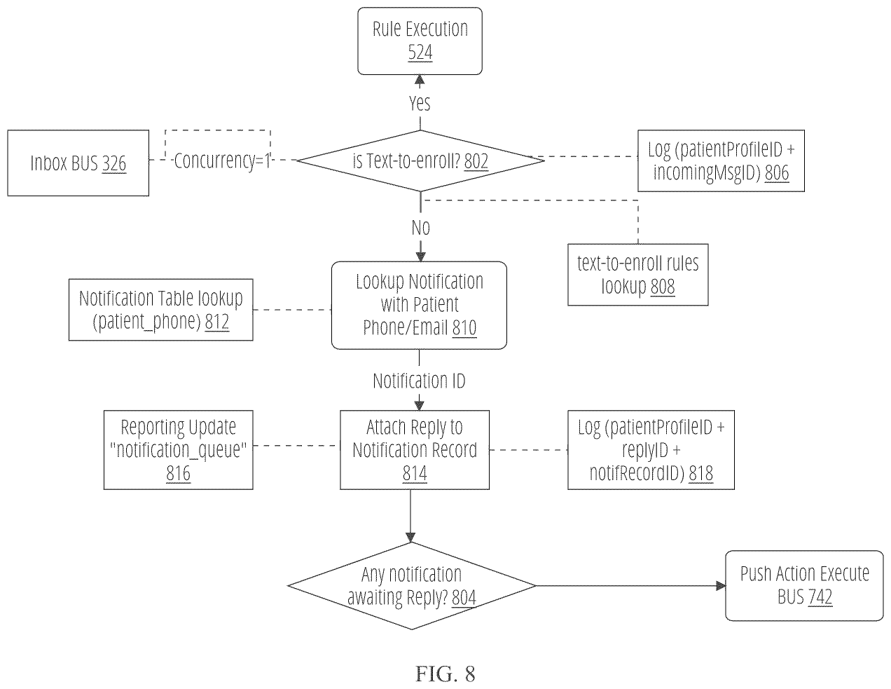

FIG. 8 illustrates the algorithmic workflow for personalized treatment identification and continuous engagement in accordance with embodiments of the inventive subject matter;

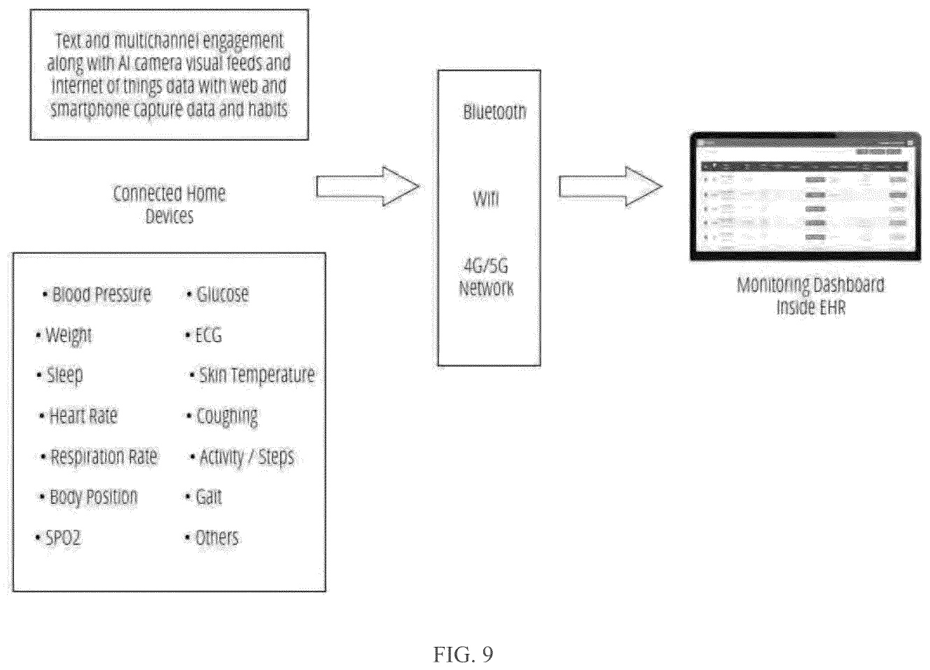

FIG. 9 illustrates the continuous monitoring strategy and feedback loop to clinicians and digital navigators used by the proposed system in accordance with embodiments of the inventive subject matter;

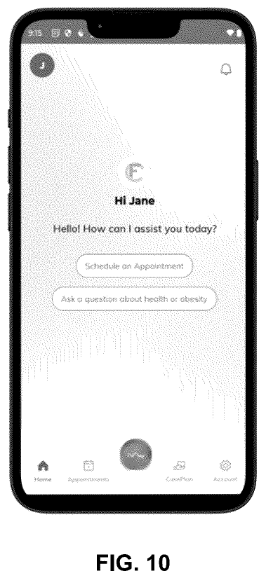

FIG. 10 illustrates the Home Dashboard of the mobile application from the patient's view, showing a greeting from AI Coach trained on obesity and medical literature and initial options in accordance with embodiments of the inventive subject matter;

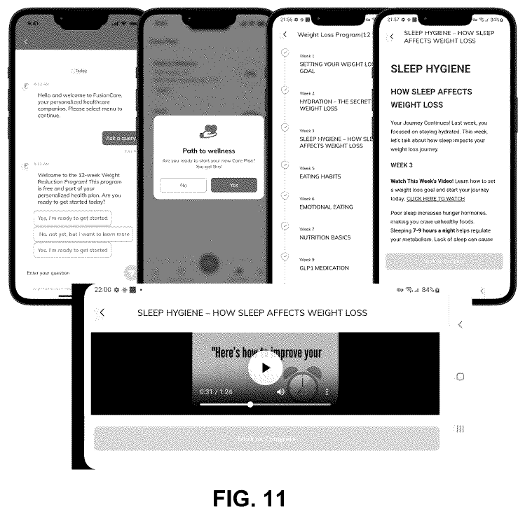

FIG. 11 illustrates various screens related to the user's care plan, including a weekly program schedule and educational content on sleep hygiene in accordance with embodiments of the inventive subject matter;

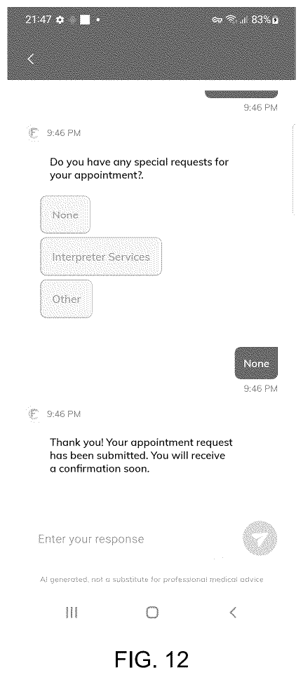

FIG. 12 illustrates a screen from the appointment booking process where a user can add special requests and receives a confirmation message in accordance with embodiments of the inventive subject matter;

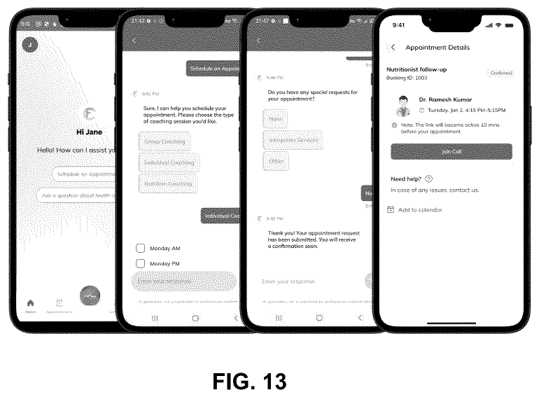

FIG. 13 illustrates the sequence of screens for booking an appointment, from the home screen to the final appointment details confirmation in accordance with embodiments of the inventive subject matter;

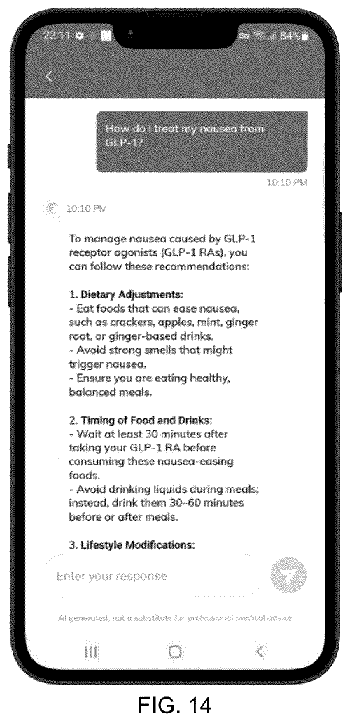

FIG. 14 illustrates a messaging screen where a user asks a question about medication side effects and receives a detailed set of recommendations from an AI coach in accordance with embodiments of the inventive subject matter; and

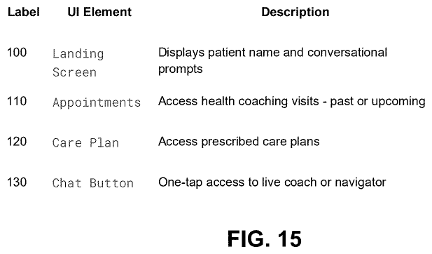

FIG. 15 is a table that describes the labels and functions of various UI elements on the application's landing screen, such as the appointments and care plan buttons in accordance with embodiments of the inventive subject matter.

### DETAILED DESCRIPTION

According to the described examples of the inventive subject matter, various apparatuses, systems, and methods using an artificial intelligence (AI) implemented engine for cardiometabolic care are provided.

Examples include a system implemented in a mobile application allowing health care professionals such as physicians to prescribe a digital health companion to their patients which can be used as part of a one-stop shop for comprehensive obesity, cardiometabolic and lifestyle care. In these examples or embodiments, the mobile application features interactive AI coaching to manage obesity and other chronic diseases. These examples include virtual health coaching that is integrated with physician-approved digital care plans to help nudge patients to take various actions and to create data-driven feedback.

In many of the embodiments, a care plan module can be a software component responsible for generating, presenting, and updating personalized treatment plans comprising scheduled tasks, educational content, and behavior-change interventions. This module interfaces with both patient-facing applications and clinician dashboards.

As used herein, including in the claims, the term “and/or,” used in connection with a list of items or categories, means one or more of the items or categories in the list, i.e., at least one of the items or categories in the list, but not necessarily all the items in the list and not necessarily one item from each category in the list. As used herein, including in the claims, the term “or,” used in connection with a list of items or categories, means one or more of the items or categories in the list, i.e., at least one of the items or categories in the list, but not necessarily all the items in the list and not necessarily one item from each category in the list. “Or” does not mean “exclusive or,” and “or” does not mean “at least one from each (category).”

The following detailed description outlines specific embodiments of the disclosed systems and methods for AI-powered personalized weight management, with reference to FIGS. 1-15 of the accompanying drawings, which illustrate representative components and workflows and are not intended to limit the scope of the claimed subject matter.

FIG. 1 shows a high-level overview of an AI-powered bot configured to leverage a curated database of clinical literature and health data to provide evidence-based guidance and dynamic education content to users. This forms the basis of the recommendation engine referenced throughout the claims, which uses supervised learning algorithms to propose tailored treatment options.

FIGS. 2-8 depict the algorithmic workflow that underpins the rules engine and feedback loop engine disclosed in the claims. For example, FIG. 2 introduces the notification architecture, wherein an event scheduler (element 302) initiates rule-based workflows that pass through various data and messaging buses. FIG. 3 illustrates how action instances are created and logged (element 504), while FIG. 4 describes branching logic in rule execution via the RxTrigger module (element 314). These figures support the claims related to asynchronous, queue-based processing.

As used with many of the embodiments, a feedback loop engine can be a system component that continuously collects patient data from one or more sources (e.g., wearables, app usage, biometric inputs) and dynamically adjusts care plans or treatment pathways based on rule-based or AI-based evaluations of such data.

As used herein, the term “RxTrigger” refers to a rule-execution initiation module or software function configured to evaluate predefined conditions and trigger the execution of corresponding actions within a care management rules engine. These actions may include, but are not limited to: (i) generation or scheduling of patient notifications, (ii) execution of care plan elements, (iii) triggering clinical decision support actions, or (iv) routing data to other workflow engines. The RxTrigger may operate in response to real-time events, scheduled workflows, user inputs, or system-generated events. The prefix “Rx” refers to prescription or treatment actions and is not limited to pharmacologic prescriptions.

FIG. 5 further expands the decision logic for when to prepare notification content (element 522), while FIG. 6 shows how immediate and scheduled notifications are handled through parallel decision paths. FIG. 7 and FIG. 8 present patient profile matching logic and message processing, which back the claimed onboarding module and hybrid care network capabilities.

As used with many of the embodiments, a hybrid care network can be a network of healthcare providers offering services through both in-person and telehealth modalities, integrated with the digital care platform to enable coordinated treatment and data-sharing across physical and virtual environments.

FIG. 9 illustrates the feedback loop engine wherein biometric data, user interactions, and provider input are synthesized into clinical insights that adapt the patient's care plan over time. These adaptations are logged and visible to both provider and patient, ensuring continuous alignment with clinical objectives.

The mobile interface, depicted in FIGS. 10-13, forms part of the patient interface described in the claims. FIG. 10 and shows a home screen with access to educational content, care plans, and chat services. FIG. 11 presents an example of an active care plan, structured into weekly behavioral and clinical tasks. FIGS. 12 and 13 illustrate the scheduling interface, allowing patients to coordinate one-on-one or group coaching sessions.

FIG. 14 presents the patient-coach messaging platform that supports behavioral nudging and context-aware guidance. This interaction channel is integral to the human-in-the-loop feature claimed. FIG. 15 provides a labeled breakdown of user interface elements referenced in FIGS. 10-13.

The system integrates an AI recommendation engine, described in the claims, trained on a curated corpus of clinical data, PubMed abstracts, and de-identified patient data from electronic health records. The training data is structured using standard clinical terminologies (e.g., SNOMED CT, ICD-10), and outputs are scored using a weighted function that ranks treatment options by predicted effectiveness and alignment with user preference and adherence history.

The feedback loop engine uses inputs such as wearable device data, patient-reported adherence metrics, electronic health records and provider-reported milestones to provide 24/7 monitoring and precision-based nudges and next best steps of action for an individual patient using both generative AI and traditional AI including rule based decision support. It includes a decision layer that compares adherence data against threshold rules to determine if coaching escalation is required or if the care plan should adapt (e.g., simplifying routines, escalating clinical interventions, or promoting new educational modules).

The rules engine described in FIGS. 2-5 is a modular component that operates on scheduled tasks, trigger events (e.g., missed check-ins), and user-specific conditions (e.g., BMI thresholds). The engine defines care plan tasks, messages, and escalation actions. Each rule includes a condition set, action set, and escalation tier, and executes through a queue-based dispatcher.

In many of the embodiments, the rules engine can be a configurable software module that executes predefined rules based on incoming data, scheduled tasks, or trigger events. Each rule may consist of one or more condition-action pairs, escalation protocols, and task definitions, and may be executed asynchronously using a queue-based dispatcher.

The onboarding module, illustrated through the screening workflows in FIGS. 7-8, dynamically generates patient profiles by parsing answers from a structured bot screener. The screener is configured to dynamically render questions based on previous responses using logic trees and adapts recommendations in real time.

The hybrid care network and marketplace features integrate with the recommendation engine to suggest interventions that match the patient profile with available in-network services. These include telehealth or brick and mortar providers and circle of care team including but not limited to advanced practice providers, nutritionists, health coaches, digital navigators, exercise physiologists, cardiac rehabilitation specialists, clinical trial recruiters, and behavioral health specialists.

All of these features, taken together, demonstrate how the claimed subject matter delivers a robust, adaptive, and personalized weight management program using machine learning, automated messaging, and clinician oversight.

One specific example of the AI Coach is a 24 hour a day/365 days a year health companion that provides instant, personalized support leveraging a protected, clinically curated knowledge base using evidence-based studies to offers immediate answers to health questions through a messaging feature. In this example, the AI Coach can answer questions and conversationally engage patients according to their personal medical journey, from medication guidance to emotional support. The AI draws from clinically curated content approved by medical professionals, ensuring all information aligns with physician recommendations. As the patient interacts with the coach, it learns preferences and challenges, continuously refining its guidance to match the user's unique health journey.

Many embodiments create structured, easy-to-follow care plans that break down complex medical recommendations into manageable daily activities. These care plans integrate:
Medication side-effect monitoring;
Nutrition guidance tailored to dietary needs and preferences;
Physical activity recommendations appropriate for current fitness levels;
Sleep optimization strategies; and
Behavioral health support for sustainable change.

The care plans can be adapted over time based on progress and feedback. This ensures the guidance remains relevant throughout a patient's health journey.

### Virtual Health Coaching

Integrated Health Coaching that seamlessly works in coordination the AI Coach and physician referred care plans to amplify personalized support between clinical visits. These professionals review progress, answer your questions, and help the patient overcome obstacles through:
Scheduled video or phone consultations;
Secure text messaging;
Progress reviews and goal adjustments; and
Specialized expertise in nutrition, exercise, behavioral health, metabolic associated complications including liver diseases, cardiovascular diseases and medication management.

Appointments for individual or group coaching are made directly through the app using the AI Coach feature, with automated reminders to keep care on track.

### Educational Content

Receive bite-sized, actionable health education twice weekly, carefully curated to support your specific health goals. The educational content focuses on:
Understanding metabolic health in a way that's relevant;
Practical nutrition and meal planning strategies;
Effective physical activity approaches;
Information to help optimize medication persistence, adherence, and outcomes; and
Behavioral techniques for forming sustainable habits.

These embodiments are designed to provide interactive challenges and activities to apply health concepts in practical ways.

### Progress Tracking and Insights

The data aggregated across the app and platform are integrated in platform progress reporting that is shared with the healthcare provider to facilitate more satisfying clinical visits and treatment.

The inventive subject matter bridges the gap between clinical visits, transforming healthcare into continuous health support. By combining the accessibility of AI with the expertise of health professionals and the structure of evidence-based care plans, we help patients implement daily behaviors to make lasting health improvements.

### Algorithm for Personalized Treatment Identification

The invention utilizes a proprietary algorithm that takes into account multiple variables such as the patient's age, weight, medical history, beliefs, prior efforts and lifestyle factors. The algorithm analyzes these variables against a database of evidence-based treatment options including pubmed and large medical databases to identify the most suitable choices for the individual. The algorithm is designed to work seamlessly with an online or bot screener, enabling real-time personalized recommendations, guided education, motivation and matching to a marketplace of proven solutions. Marketplace also includes a national network of physicians who are trained in obesity medicine and are onboarded in “hybrid practice” concept.

### System for Continuous Engagement

The proposed system employs a multi-modal approach for continuous patient engagement. Data from wearable devices, mobile applications, and coaching services are integrated into a centralized platform. The system then uses AI-driven analytics to interpret this data, allowing for dynamic adaptation of treatment plans. The platform also includes features for automated follow-ups, reminders, and feedback, ensuring that patients remain committed to their weight management journey.

### Human in the Loop

The automation is backed by “human in the loop” digital care coaches, navigators, nutritionists, psychologists, and other care team personnel to augment work from AI and generative AI with human decision-making and coaching interaction.

In one embodiment, the system identifies and enrolls candidate patients in clinical trials by matching diagnosis codes, adherence behavior, geographic data, and biometric trends. Trial outcomes are stored and analyzed to generate real-world evidence and refine treatment matching algorithms.

In another embodiment, the system includes an AI model configured to generate an adherence likelihood score for each user, based on historical treatment engagement, behavioral risk factors, and sociodemographic variables. The AI engine uses this score to prioritize interventions more likely to be successful.

In some configurations, if biometric or adherence data indicates a deviation from clinical targets, an automated escalation workflow is triggered. This prompts a human care provider to intervene, confirm the plan, or adjust interventions manually.

In an alternate embodiment, educational modules are presented in adaptive sequences based on prior comprehension indicators, such as quiz scores, screen time, and message response tone. Users flagged for difficulty receive simplified tracks or coaching referrals.

Additionally, the platform incorporates a curated marketplace of third-party services including telehealth, fitness, sleep, and nutrition solutions. Post-intervention data is fed back into the recommendation engine, creating a learning loop that enhances precision of future suggestions.

In one use case example, Jane Doe, a 48-year-old with metabolic syndrome, is onboarded using a screener bot. Based on her history, wearable data, and preferences, the system assigns a tailored care plan with GLP-1 therapy, behavioral nudges, and sleep optimization. Two weeks in, low adherence triggers human review. Her coach modifies the plan to increase flexibility and engagement. Over 90 days, Jane's key biometrics improve 24% from baseline.

Many embodiments include one or more algorithms for identifying evidence-based treatment options based on individual parameters combined with a platform for continuous patient engagement. In one example, the algorithm utilizes variables such as age, weight, medical history, and lifestyle factors and matches them to a marketplace of proven weight loss solutions and physicians network enrolled in hybrid practice. The platform can integrate data from one or more types of devices and/or services, for example wearable devices, mobile applications such as Apple® Heath application, and one or more coaching algorithms or services. The platform may use AI-driven analytics to adapt treatment plans dynamically. The platform may use AI-driven analytics to recruit patients into clinical trials and generate real world evidence and digital biomarkers predicting which patients respond to which treatment/s over short and long term.

FIG. 1 illustrates the AI-powered BOT using evidence from hundreds of thousands of published literature and guided education and recommendations.

FIG. 2 through FIG. 15 illustrate details of the algorithmic workflow for personalized treatment identification and continuous engagement and exemplary output screens from the platform. In one example, the system includes Notification sender 202 which communicates with multiple channels such as Twilio (SMS) 204, sendgrid (email) 206, voice based conversation AI agent, app based notifications and IVR 208.

The system start points are daily prep scheduler (S1) 302, rule execution request (single/bulk) 304, an incoming notifications/SMS 306, and notification send scheduler (S2) 308. The daily prep scheduler (S1) 302 communicates through HTTP to the fetch next-day Notifications to be prepped (status=‘prep_queued’) 312 which performs an Action Instance Table lookup (effectiveDate+status=‘queued’) 310, and a function call to the RxTrigger 314 and the DB BUS 322. The rule execution request (single/bulk) 304 communicates via HTTP to the RxTrigger 314. The incoming notifications/SMS 306 communicates with the RxTrigger 314 via a function call. The notification send scheduler (S2) 308 communicates with the RxTrigger 314 and performs Log (Event ID+Published Date) 316. The RxTrigger 314 moves to determine request type? 318. If the request type is action execute moves to action Execute Bus 320. If the action is a prescription, it moves to the Rule execution BUS 324. If the action is an incoming message, it moves to the inbox BUS 326 if the action is a notification send, it moves to Notification BUS 328.

DB BUS 322 moves with a concurrency=1 to the DB operation 402 which performs Reporting CRUD 404. The Notification BUS 328 moves with a concurrency=S0 to the notification sender 406 which performs Log (patientProfileID+rulesInstanceID+notifRecrodID+sendDetails) 408, and send details to perform Update Notification Record (staus=‘success’+Send Details) 410 which also performs Log (patientProfileID+rulesInstanceID+notifRecordID) 412 and Reporting Update “notification_queue” 414.

The action Execute Bus 320 moves with a concurrency=SO to the decision is EffectiveDate before next Prep Time? 502. If No, Create Action Instance Record (status=‘queued’+effectiveDate only) 508 is performed which also performs Reporting Create “action_instance” 504 and Log (patientProfileID+rulesInstanceID+actionInstanceID) 506. If yes, Action Definition Lookup 510 is performed and moves the action definition to Create Action Instance if required 512 which performs Log (patientProfileID+rulesInstanceID+actionInstanceID) 514, and Reporting create “action_instance” 516. The action instance ID is handed to the Parent/Child node processor 518 which determines switch Action type 520. If it is a sub-rule, then Rule Execution 524. If it is a notification, then Prepare Notification content 522.

From Prepare Notification content 522, is notification immediate (SYNC) 602 determination is made. If no, then the Create Notification Record (status=‘queued’+Notif Content) 604 is performed while also Reporting Create “notification_queue” 612. Create Scheduler (S2) Instance for Notification 606 is scheduled while Log (PatientProfileID+ruleInstanceID+notifRecordID) 608 is performed. Following Create Scheduler (S2) Instance for Notification 606, Log (PatientProfileID+ruleInstanceID+s2InstanceID) 610 is performed. If yes, then Create Notification Record (status=‘queued’) 614 is performed with Reporting Create “notification_queue” 612 while performing Log (PatientProfileID+ruleInstanceID+notifRecordID) 616. After Create Notification Record (status=‘queued’) 614, process moves to Notification Sender 618 which performs Log (PatientProfileID+ruleInstanceID+notifRecordID+sendDetails) 620 and sends details to Update Notification Record (status=‘success’+send details) 622 which performs Report Create “action_instance” 626 and Log (PatientProfileID+ruleInstanceID+notifRecordID) 624.

Rule execution BUS 324 communicates with the Rule Execution 524 with a concurrency=SO. The Rule Execution 524 performs Facility Context lookup (notification sender, timezone, etc.) 702 and communicates with Patient Profile Matching Algorithm 704 which performs Patient Profile Lookup (key: facility+MRN OR demographics) 706. The Patient Profile Matching Algorithm 704 determines patient found? 708. If no, then Create Patient Profile 712 while performing Reporting Create “patient_profile” 716 and providing the patient profile ID. If yes, then Rule Design/Infor Lookup (Actions IDs & schedule) 718 with the patient profile ID from the Create Patient Profile 712 while performing Log (patient profile ID+event ID) 720. After Rule Design/Infor Lookup (Actions IDs & schedule) 718, Create Rules Instance 722 is performed while also performing Reporting Create “rules_instance” 726 and Log (patientProfileID+rulesInstanceID) 724. The Create Rules Instance 722 provides rules instance ID to Loop over Action (A1 . . . . An) 728 which determines is action left? 732. If no, then Tag Rule Instance to Patient 734 which also performs Log (patientProfileID+rulesInstanceID) 736 and Reporting Update “patient_profile” 738. The process then moves to Execution End 740. If yes and provided with a rules instance ID, the proces moves to the Push Action Execute BUS 742 which performs Log (patientProfileID+replyID+actionDefID) 744.

The inbox BUS 326 operates with a concurrency=1 the determination of is Text-to-enroll? 802 while also performing Log (patientProfileID+incomingMsgID) 806. If yes, then the process moves to the Rule Execution 524. If no, the process moves to the Lookup Notification with Patient Phone/Email 810 while also performing a text-to-enroll rules lookup 808. During the Lookup Notification with Patient Phone/Email 810 a Notification Table lookup (patient_phone) 812 is performed and the notification id is provided to in order to Attach Reply to Notification Record 814 which also performs Reporting Update “notification_queue” 816 and Log (patientProfileID+replyID+notifRecordID) 818. The process then determines Any notification awaiting Reply? 804 which moves to the Push Action Execute BUS 742.

FIG. 9 illustrates the continuous monitoring strategy and feedback loop to clinicians and digital navigators used by the proposed system.

While the present application discusses embodiments for providing healthcare systems and methods, the exemplary embodiments discussed herein may be used in conjunction with other physical sensors, relay or services as well. Recommendation systems and methods discussed herein, may be implemented or used to modify the algorithms. Some embodiments of the present inventive subject matter may also be used in conjunction with other products and services such as other types of health monitoring or feedback systems and methods. Now that embodiments of the present inventive subject matter have been shown and described in detail, various modifications and improvements thereon can become readily apparent to those skilled in the art. Accordingly, the exemplary embodiments of the present inventive subject matter, as set forth above, are intended to be illustrative, not limiting. The spirit and scope of the present inventive subject matter is to be construed broadly.

What is claimed is:

1. A system for personalized weight management, comprising:
a patient interface configured to present educational content and receive patient responses;
a recommendation engine comprising an AI model trained on clinical data to identify personalized treatment options based on patient-specific parameters;
a care plan module configured to generate and update individualized care plans;
an engagement module configured to receive inputs from wearable devices and track adherence;
a feedback loop engine configured to adapt the care plan in response to adherence data, clinical goals, and user feedback;
a communication module configured to transmit one or more of the following: reminders, educational messages, and alerts via SMS, email, conversational AI and in-app messaging;
a coach interface configured to allow human healthcare providers to intervene or override AI-generated recommendations;
a rules engine utilizing generative AI powered guidance that schedules and triggers care plan actions based on time, activity, and patient condition;
a data repository configured to store user profiles, progress logs, and treatment history;
wherein the system is configured to continuously adapt care recommendations using a combination of AI-based analysis and human review.

2. The system of claim 1, wherein the AI model is trained on a curated database of peer-reviewed literature and clinical trial data.

3. The system of claim 1, wherein the engagement module includes a notification scheduler that triggers at least one or more of the following: SMS, IVR, conversational AI agent and in-app message.

4. The system of claim 1, wherein the recommendation engine is further configured to stratify treatment options by predicted efficacy and patient adherence likelihood.

5. The system of claim 1, wherein the care plan module supports goal-setting and automated progress monitoring.

6. The system of claim 1, wherein the system integrates a patient-to-coach chat interface for contextual questions and behavioral nudging.

7. The system of claim 1, wherein the feedback loop engine is configured to modify treatment recommendations in real-time based on collected biometric data.

8. The system of claim 1, wherein the rules engine is implemented as a queue-based workflow engine supporting concurrent asynchronous triggers.

9. The system of claim 1, wherein the system includes an onboarding module configured to dynamically generate patient profiles using a bot-based screener.

10. The system of claim 1, wherein the system is further configured to identify candidate patients for clinical trial enrollment based on collected real-world data, user profile matching and treatment outcomes.

11. A method of delivering AI-driven personalized weight management, comprising: receiving, via a bot screener, patient-specific input data including medical history, goals, and prior interventions;
identifying a plurality of evidence-based treatment options using an AI recommendation engine;
generating a personalized care plan comprising scheduled interventions, behavioral goals, and educational content;
presenting the care plan to the patient through a mobile application;
collecting adherence and biometric data via connected devices and patient input;
evaluating collected data using a feedback loop engine;
modifying the care plan based on adherence metrics, feedback, and treatment response;
generating automated reminders and motivational messages via multiple communication channels;
allowing healthcare providers to review, modify, or override care plan elements;
storing the interaction data and treatment outcomes in a patient record database.

12. The method of claim 11, wherein the AI recommendation engine uses a weighted scoring algorithm to rank treatment options.

13. The method of claim 11, wherein modifying the care plan comprises adjusting medication reminders, nutritional goals, or behavioral challenges.

14. The method of claim 11, further comprising identifying treatment-resistant patients for alternate intervention.

15. The method of claim 11, further comprising presenting educational content in personalized sequences based on comprehension or engagement level.

16. The method of claim 11, wherein adherence is calculated based on thresholds for data reporting frequency, user input completeness, or biometric trend conformity.

17. The method of claim 11, further comprising triggering human coach intervention when adherence falls below a configurable threshold or one or more other triggers for human in the loop intervention.

18. The method of claim 11, further comprising matching patients to healthcare providers within a hybrid care network.

19. The method of claim 11, wherein the bot screener presents adaptive questions based on initial user responses.

20. The method of claim 11, further comprising automatically tagging users for clinical trial eligibility based on system-collected outcome metrics.
